(19) United States
(12) Patent Application Publication
(10) Pub. No.: US 2020/0373006 A1
(43) Pub. Date: Nov. 26, 2020

(54) MEDICAL INFORMATION PROCESSING SYSTEM

(71) Applicants: NEC Corporation, Tokyo (JP); KITAHARA MEDICAL STRATEGIES INTERNATIONAL CO., LTD., Tokyo (JP)

(72) Inventors: Masahiro KUBO, Tokyo (JP); Masahiro HAYASHITANI, Tokyo (JP); Yuji OHNO, Tokyo (JP); Toshinori HOSOI, Tokyo (JP); Shigemi KITAHARA, Tokyo (JP)

(73) Assignee: NEC Corporation, Tokyo (JP)

(21) Appl. No.: 16/645,248

(22) PCT Filed: Sep. 3, 2018
(86) PCT No.: PCT/JP2018/032565

§ 371 Date: Mar. 6, 2020

(30) Foreign Application Priority Data
Sep. 8, 2017 (JP) 2017-172847

(51) Int. Cl. G16H 40/20; G06Q 10/10; G16H 50/20; G06Q 10/04; G16H 70/20; G06Q 30/02; G16H 50/70

(52) U.S. Cl. CPC G16H 40/20; G06Q 10/10; G16H 50/20; G16H 50/70; G16H 70/20; G06Q 30/0205; G06Q 10/04

(57) ABSTRACT

This medical information processing system is provided with: an operation policy input unit which receives a selection of an operation policy defining a plurality of operation policies which can be taken by a hospital as a whole; a model learning unit which refers to a database being managed so as to be accessible in the hospital, and generates, for each selectable operation policy, a maximization model of each operation policy with respect to an environmental change of the hospital; and a behavior optimization unit which, using the maximization model of the operation policy selected by the operation policy input unit, generates decision assistance information for a healthcare worker with respect to each patient that maximizes the overall efficiency of the hospital in accordance with the environmental change of the hospital.

### TECHNICAL FIELD

This invention relates to a medical information processing system, a method of generating decision support information, and a program therefor, which are to be implemented in a medical institution including a hospitalization facility.

### BACKGROUND ART

Recently, many information processing systems have been used in medical institutions, and information processing systems specifically designed for medical institutions have also been actively developed.

At medical institutions, a wide range of operational tasks including surgery, examinations, and rehabilitation are performed on many patients. The information processing systems specifically designed for medical institutions support the former operational tasks performed by medical personnel and improve work efficiency.

Examples of the information processing systems specifically designed for medical institutions include systems configured to convert a previous paper-based medical chart into an electronic medical chart, and systems configured to receive medical chart information as electronic data from the beginning. An electronic medical chart information group of each patient is stored in a server (storage) of the information processing system, and is called by an authorized staff member of the medical institution to be used as necessary in the same manner as the previous paper-based medical chart. An information processing system that handles only the collection and presentation of electronic medical charts is generally called an electronic medical chart system.

The medical information processing systems used in medical institutions are not limited to electronic medical chart systems. Examples of a wide variety of such medical information systems include one system described in Patent Literature 1.

In Patent Literature 1, there is described a medical support system for determining medical care for an observer. The medical support system causes a processor to execute the steps of: acquiring observer data; evaluating a clinical need of the observer; proposing a clinical outcome; and determining, based on a service/outcome/needs model, a service to be provided to one patient in order to achieve the clinical need and the proposed clinical outcome.

In a method using a medical support system described in Patent Literature 1, a plurality of computer models are obtained through machine learning performed on various information obtained from inside and outside the hospital, to thereby be able to determine the service for the patient that meets the clinical need and the proposed clinical outcome based on the plurality of models.

In addition to Patent Literature 1, medical information processing systems used in medical institutions are also described in, for example, Patent Literature 2 to Patent Literature 6.

In an integrated medical backbone work system described in Patent Literature 2, a mechanism for acquiring patient attributes and the like and generating a comprehensive electronic medical chart, a mechanism for supporting medical diagnosis and treatment and the like by a doctor and the like, and an integrated backbone database for recording medical information on individuals are connected to one another by a network, to thereby integrate a medical environment at a hospital, a home, and the like.

In a medical diagnosis-and-treatment support system described in Patent Literature 3, when medical diagnosis and treatment on a patient are performed, preparation of a plan therefor and confirmation of past and current situations are facilitated by arranging an index screen displaying a medical diagnosis-and-treatment record index and a medical diagnosis-and-treatment plan screen displaying a medical diagnosis-and-treatment plan table of the patient next to each other.

In a medical information processing system described in Patent Literature 4, during a disaster, the disaster situation for the near future and the situation facing a medical diagnosis-and-treatment institution are predicted based on a disaster situation changing moment by moment and progress of handling being made by the medical diagnosis-and-treatment institution, to thereby determine whether or not to transfer patients and where to place medical personnel and equipment.

In a medical management support system described in Patent Literature 5, a project network diagram for each patient are prepared and displayed, to thereby promote the execution support of various work at a medical institution, the improvement of the quality of medical care, and the improvement of the administration of the medical institution.

In an analysis system described in Patent Literature 6, an alleviation period of side effects is calculated from changes in examination results, and information on drugs causing side effects, which indicates a relationship between the side effects and the drugs other than the drugs that have been prescribed continuously since the start of the alleviation period among the drugs being prescribed, is calculated, to thereby produce the information on the drugs relating to the side effects.

### CITATION LIST

### Patent Literatures

PTL 1: JP 2016-520941 A

PTL 2: JP 2002-056093 A

PTL 3: JP 2004-021380 A

PTL 4: JP 2005-346589 A

PTL 5: JP 2007-140607 A

PTL 6: WO 2016/103322 A

### SUMMARY OF INVENTION

### Technical Problem

The above-mentioned technology described in Patent Literature 1 includes the step of using a service/outcome/needs model created by collecting observer data and by machine-learning to determine a service to be provided to one patient for a clinical need and a proposed clinical outcome.

Therefore, the medical support system, by obtaining a plurality of computer models through machine learning for various information obtained from inside and outside the hospital, thereby is able to determine the service for one patient that meets the clinical need and the proposed clinical outcome based on the plurality of models.

However, in the technology described in Patent Literature 1, for example, there can be pointed out improvement to be achieved in optimizing issues across a plurality of patients. In other words, with the medical support system of Patent Literature 1, there may arise a case in which deriving the service to be provided to one patient prevents the optimal service from being provided to another patient.

Meanwhile, in Patent Literature 5, there is disclosed a system for optimizing resources with respect to a plurality of patients by accumulating tasks. However, a method based on the accumulation of tasks is weak at handling information to be supplied in the future, and there is a high possibility that the entire schedule may be greatly disrupted due to, for example, the occurrence of an emergency patient.

In addition, many hospitals are required to handle various changes every day. A hospital director and a person in charge of each management department manage the hospital under such various changes in accordance with various operation policies. Those operation policies are subject to change for some reason. For example, it can be assumed that an operation policy is changed when an unexpected event occurs or an environment outside the hospital changes.

Meanwhile, the technology described in Patent Literature 1 and the technology described in the other Patent Literatures cannot handle changes in operation policy.

It is an object of this invention to provide a medical information processing system for solving the problems described above by supporting decisions by medical workers with respect to each patient based on the selected operation policy information on the entire hospital and a database reflecting the current environment inside and outside the hospital.

### Solution to Problem

A medical information processing system according to one embodiment of this disclosure comprises an operation policy input unit configured to receive a selection of an operation policy in which a plurality of management policies takable by an entire hospital are defined; a model learning unit configured to generate, for each selectable operation policy, a maximization model in response to a change in an environment of the hospital for each operation policy by referring to a database managed so as to be accessible at the hospital; and a behavior optimization unit configured to generate, through use of the maximization model for the operation policy selected by the operation policy input unit, decision support information for a medical worker with respect to each patient in order to maximize overall efficiency of the hospital in accordance with the change in the environment of the hospital.

A determination support information generation method according to one embodiment of this disclosure comprises preliminarily generating, by a model learning unit, for each of a plurality of operation policies takable by the entire hospital, a maximization model in response to a change in an environment of a hospital for each operation policy by referring to a database managed so as to be accessible at the hospital; receiving, by an operation policy input unit, a selection of an operation policy; and generating, by a behavior optimization unit, decision support information for a medical worker with respect to each patient in order to maximize overall efficiency of the hospital in accordance with the change in an environment of the hospital through use of the maximization model for the selected management policy.

A program, according to one embodiment of this disclosure, causing a processor of an information processing system to operate as an operation policy input unit configured to receive a selection of an operation policy in which a plurality of operation policies takable by an entire hospital are defined; a model learning unit configured to generate, for each selectable operation policy, a maximization model in response to a change in an environment of the hospital for each operation policy by referring to a database managed so as to be accessible at the hospital; and a behavior optimization unit configured to generate, through use of the maximization model for the operation policy selected by the operation policy input unit, decision support information for a medical worker with respect to each patient in order to maximize overall efficiency of the hospital in accordance with the change in an environment of the hospital.

### Advantageous Effects of Invention

According to this invention, the medical information processing system configured to support decisions by medical workers with respect to each patient based on the selected operation policy information on the entire hospital and the database reflecting the current environment inside and outside the hospital can be provided.

### BRIEF DESCRIPTION OF THE DRAWINGS

FIG. 1 is a block diagram for illustrating a medical information processing system 1 according to an example embodiment of this invention.

FIG. 2 is a flowchart for illustrating a basic flow of the medical information processing system 1 according to the example embodiment of this invention.

FIG. 3 is a flowchart for illustrating a schematic machine learning flow of the medical information processing system 1 according to the example embodiment of this invention.

FIG. 4 is a flowchart for illustrating a decision support information generation routine of the medical information processing system 1 according to the example embodiment of this invention.

FIG. 5 is an explanatory diagram for visually illustrating an operation of the medical information processing system 1 according to the example embodiment of this invention.

FIG. 6 is a block diagram for illustrating a configuration example of the medical information processing system according to this invention.

FIG. 7 is a block diagram for illustrating another configuration example of the medical information processing system according to this invention.

### DESCRIPTION OF EMBODIMENTS

Example embodiments of this invention are now described with reference to the drawings.

### EXAMPLE EMBODIMENTS

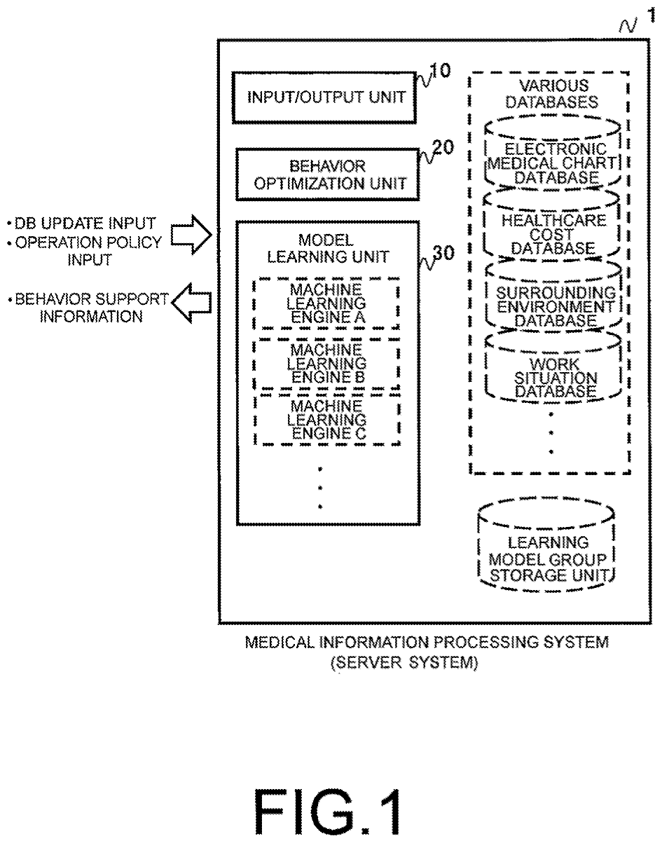

FIG. 1 is a block diagram for illustrating a medical information processing system 1 according to an example embodiment of this invention.

The medical information processing system 1 includes at least an input/output unit 10, a behavior optimization unit 20, and a model learning unit 30. In the medical information processing system 1, it is assumed that there are constructed various databases in which respective components are available as required and a learning model group storage unit configured to store learned learning models. The various databases to be used may each be an internal database or an external database. Similarly, the learning model group storage unit to be used may be an external database and the like instead of being an internal memory or an internal database. The medical information processing system 1 includes a processor and a memory, and operates the respective components as follows.

The input/output unit 10 is an input/output interface, and operates as operation policy input means to receive a plurality of operation policies (pieces of operation policy information) that are takable by the entire hospital. The operation policies that may be selected as options are, for example, indices that contribute to improvement in hospital management or to more efficient work in terms of hospital operation. Examples of the operation policies include, but are not particularly limited to, a cost reduction index for the entire hospital, a power saving enhancement index for the entire hospital, a hospitalization time minimization index for all of a large number of patients, and a working hour minimization index for the hospital staff. The operation policies may be set by, for example, a hospital director or a manager.

The input/output unit 10 sequentially receives electronic medical chart information on each patient and various information from inside and outside the hospital, and sequentially registers the received information in a database. The input/output unit 10 also acquires and registers various information from inside and outside the hospital from an external database.

The behavior optimization unit 20 reads, from the learning model group storage unit, a maximization model for the selected operation policy via the input/output unit 10, and generates decision support information for medical workers with respect to each patient by referring to various databases for various information to be used. The decision support information for the medical workers is information for supporting the behaviors of each medical worker in order to maximize the selected operation policy for the entire hospital. The decision support information is appropriately generated automatically or in response to a user request in accordance with a change in an environment of the hospital at that point in time by the maximization model. In other words, the decision support information may be appropriately generated by the behavior optimization unit 20 automatically or in response to a user request in accordance with the current situation of the various databases by the maximization model.

The behavior optimization unit 20 may generate a notification indicating a change policy of a hospital room environment with respect to each patient as the decision support information. As a result of this adjustment of the hospital room environment, the activation of a natural healing effect can be achieved. As the adjustment items of the hospital room environment, it is desired to include, for a hospitalized patient, a hospital room brightness, a room odor, a sound (including environmental music, background music, and silence), a room temperature, and a humidity. A medical worker who has received the decision support information considers whether or not to change each hospital room environment in accordance with the patient (group), and changes the hospital room environment as required.

Similarly, the behavior optimization unit 20 may also generate a notification indicating an ordering policy of each patient as the decision support information. It is desired that the ordering policy include, for each patient, a prescription, an examination, and a treatment plan. A medical worker who has received the decision support information considers the ordering policy in accordance with the patient, and carries out the prescription, examination, and treatment plan based on the indicated policy as required. The treatment plan is a plan indicating, for a given patient, the procedure or order in which treatment is to be performed in regard to, for example, the administration of drugs and rehabilitation instructions. Specifically, the treatment plan is information indicating a treatment order, for example, treatment A→treatment B (or treatment B′)→treatment C.

Similarly, the behavior optimization unit 20 may also generate a notification indicating a post-discharge care work policy for each patient as the decision support information. It is desired that the post-discharge care work policy include, for each patient, transfer destination candidates (for example, convalescent hospital, various facilities, and home). A medical worker who has received the decision support information considers the transfer destinations in accordance with the patient, and carries out the work for moving the patient to a transfer destination candidate based on the indicated policy as required.

The model learning unit 30 refers to various databases managed so as to be accessible at the hospital by using various machine learning methods, such as a regression method, decision tree learning, a Bayes method, a kernel method, a neural network, and deep learning, to generate and update, for each selectable operation policy, a maximization model for the change in an environment of the hospital for each operation policy. The model learning unit 30 may also refer to various databases managed so as to be accessible at the hospital to include, for each selectable operation policy, a change in an environment of the hospital and a shift from the treatment plan with respect to each patient for each operation policy, as machine learning items. The shift from the treatment plan is, for example, a change in the treatment details of the initial treatment plan (or the cause of such change) in which the degree of recovery of the patient from the illness or injury is different from the initial treatment plan due to treatment based on the initial treatment plan.

The model learning unit 30 operates as model learning means. The items (parameter group) used for machine learning may include items to be used for learning the disease name and medical condition of each patient with reference to, for example, an electronic medical chart database. Similarly, the parameter group used for machine learning may be selected by constructing a healthcare cost database, a surrounding environment database, a work situation database, and the like in such a manner that those databases can be referred to. It is desired to provide many parameters to be included in the learning items. In the case of parameters representing the environment in relation to climate, examples thereof may include the weather, temperature, and humidity outside the hospital, the room temperature and humidity in the hospital, and the average weekly/monthly temperature. Any record information appropriately managed in a database may be used.

The parameters relating to a change in an environment of the hospital may include at least one of information on the number of inpatients at a neighboring medical institution, the number of inpatients at a transfer destination medical institution, a local climate, a local population increase or decrease, and the number of outpatients. The change in an environment of the hospital may also include, as appropriate, the fact that a competing hospital has been established, seasonal variations in the number of patients, a resource situation of the transfer destination hospital, healthcare cost data, and variations in patient questionnaire results. In particular, by including healthcare cost data in the machine learning of each operation policy, it is possible to produce a significant variation as a content difference of the decision support information for each operation policy that can be classified in terms of hospital management aspects, medical quality, medical efficiency, hospital stay time, patient satisfaction, and the like.

It is desired that the parameters relating to the treatment plan with respect to each patient include a scheduled date and an execution date each of which relates to, for example, an examination date, an informed consent date, or a discharge date as individual items. For the shift from the treatment plan with respect to each patient, the occurrence situation of emergency patients, the degree of recovery of the patient, and the shift from the treatment plan for an individual patient can be included in the learning parameters. By adding those parameters, a significant variation can be produced as a content difference of the decision support information for each operation policy that can be classified in terms of hospital management aspects, medical quality, medical efficiency, hospital stay time, patient satisfaction, and the like.

The machine learning method is not particularly limited, and there may be used an expected value maximization method, an EM method (expectation-maximization algorithm), or the like. As a reinforcement learning algorithm, for example, a regression method, for example, a support vector machine (SVM), a clustering method, for example, a k-nearest neighbor method, a neural network method, for example, learning vector quantization, an ensemble method, for example, random forest, and the like may be used in combination as required.

In this way, the medical information processing system 1 provides the medical worker with decision support information that is employable as a likely optimal behavior policy for the entire hospital in accordance with sequential adjustment to the current situation by using various databases managed so as to be accessible at the hospital and a learning model of each hospital operation policy.

It is desired that a large number of individual environments at the hospital that change and that can be input, acquired, and sensed be managed as individual items in various databases. By managing such environments, changes in an environment of the hospital, which may change from moment to moment, can be reflected in the maximization model for each operation policy and in each piece of decision support information via the model learning unit 30 and the behavior optimization unit 20.

Similarly, it is desired that shifts from the treatment plan with respect to each patient that can be input, acquired, and sensed be included and managed as individual items in various databases. By managing such shifts, a shift from the treatment plan with respect to each patient, which may occur from moment to moment, can be reflected in the maximization model for each operation policy and in each piece of decision support information via the model learning unit 30 and the behavior optimization unit 20.

With the above-mentioned configuration, the medical information processing system 1 can support decisions by medical workers with respect to each patient based on the selected operation policy information on the entire hospital and a database reflecting the current environment inside and outside the hospital.

[Description of Operation in Example Embodiment]

Next, an operation of the medical information processing system 1 according to the example embodiment will be described.

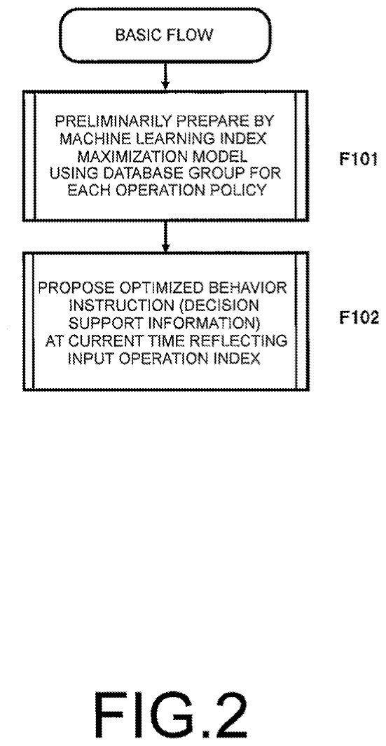

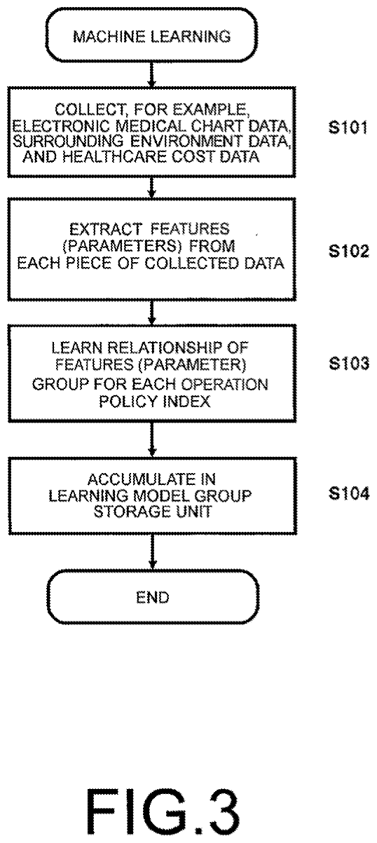

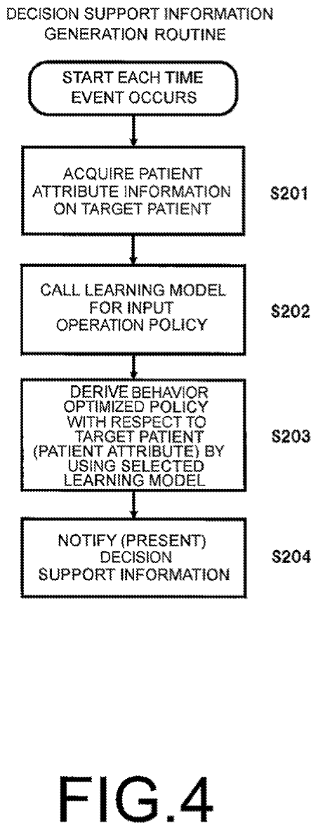

FIG. 2 is a flowchart for illustrating a basic flow of the medical information processing system 1 according to the example embodiment. FIG. 3 is a flowchart for illustrating the machine learning flow of the medical information processing system 1. FIG. 4 is an example of a flowchart for illustrating the decision support information generation routine of the medical information processing system 1.

Firstly, the basic flow is as follows as illustrated in FIG. 2.

The medical information processing system 1 causes the model learning unit 30 to preliminarily machine-learn an index maximization model for each operation policy by referring to various databases managed so as to be accessible at the hospital (Step F101).

The medical information processing system 1 causes the behavior optimization unit 20 to generate decision support information for a medical worker with respect to each patient by using the maximization model for the selected operation policy, and proposes the generated decision support information to the medical worker (Step F102).

As indicated by this flow, the medical information processing system 1 receives selection or change of the operation policy via the input/output unit 10, and provides the medical workers belonging to the various departments in the hospital with behavior support with respect to each patient that is capable of maximizing the overall efficiency of the hospital based on the latest operation policy.

This enables each medical worker to perform the work with respect to each patient with reference to the decision support information. As a result, each medical worker can perform behaviors on each patient based on the details of the behavior support information in accordance with the operation policy derived based on various information that the medical worker would not know by themselves as well as matters that the medical worker knows by themselves.

Next, FIG. 3 is an example of a flowchart for illustrating the machine learning flow of the medical information processing system 1.

First, the processor of the information processing system serving as the medical information processing system 1 sequentially collects, in various databases, a large amount of data (for example, electronic medical chart data, surrounding environment data, and healthcare cost data) to be learned (Step S101).

Next, the processor extracts data of items (characteristics and parameters) to be learned from data groups accumulated in various databases (Step S102).

Next, the processor learns the relationship of the feature (parameter) group for each operation policy index (Step S103).

Lastly, the processor accumulates the learned results in the learning model group storage unit for each index (Step S104).

It is desired that the machine learning be performed regularly and updated to the latest learned results.

FIG. 4 is an example of a flowchart for illustrating a decision support information generation routine of the medical information processing system 1. It is assumed that this decision support information is, for example, looked at by a doctor before treating the next patient to be examined. The decision support information generation routine is assumed to be performed at each place at which medical workers perform work on patients in the hospital.

First, the processor of the information processing system serving as the medical information processing system 1 acquires patient information on a target patient (Step S201). For example, the patient information may be acquired from an electronic medical chart database.

Next, the processor calls a learning model (maximization model) of the input operation policy (Step S202).

Next, the processor derives a behavior optimized policy for the target patient (patient attribute) based on the learning model of the selected index (Step S203).

Lastly, the processor notifies the medical worker of the derived behavior optimized policy as decision support information (Step S204).

The processing for generating decision support information for the medical worker with respect to the patient may be performed as appropriate in response to a request from the medical worker, or the decision support information may be automatically generated when the medical worker approaches the hospital room.

By operating the information processing system in this way, the medical information processing system 1 can support decisions by medical workers with respect to each patient based on the selected operation policy information on the entire hospital and a database reflecting the current environment inside and outside the hospital.

Now, the overall operation of the medical information processing system 1 will be described through visual illustration.

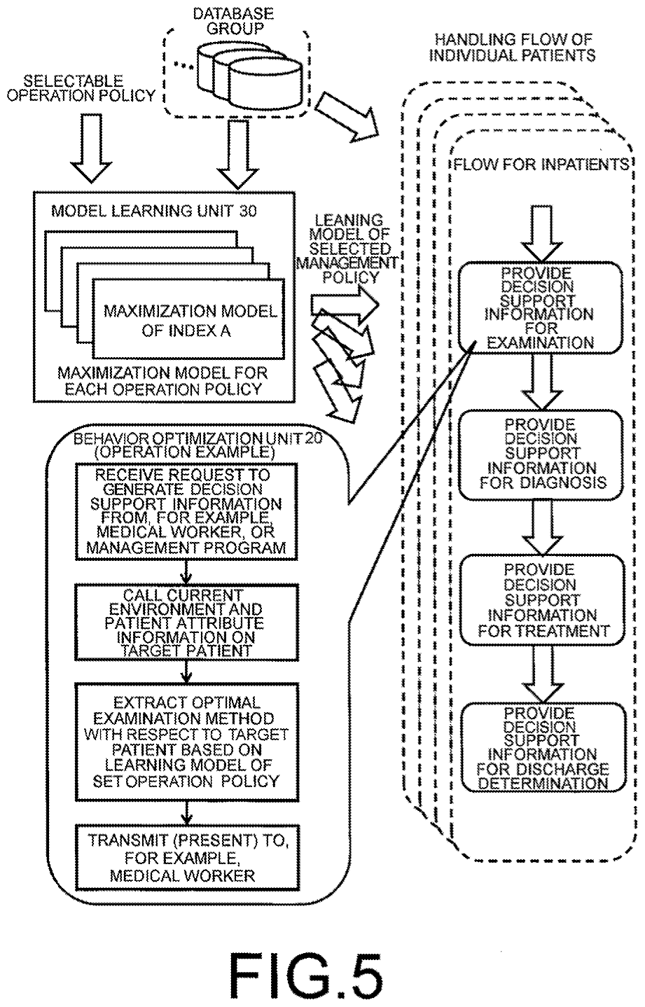

FIG. 5 is an explanatory diagram for visually illustrating the operation of the medical information processing system 1.

The illustrated flow of the handling of a patient is based on a typical flow of how an inpatient is handled in a hospital. This flow is represented as blocks until discharge from hospital, going in the order of “examination”→“diagnosis”→“treatment”→“discharge determination”. The handling flow of this individual patient may be appropriately rearranged depending on whether the patient is an outpatient, an emergency outpatient, an inpatient, and the like.

The medical information processing system 1 preliminarily generates, for each of a plurality of operation policies that are takable by the entire hospital, a maximization model (learning model) in response to a change in an environment of the hospital for each operation policy by referring to various databases managed by the model learning unit 30 so as to be accessible at the hospital. The medical information processing system 1 also performs reinforcement learning of each learning model as appropriate.

The medical information processing system 1 causes the input/output unit 10 to receive a selection of a operation policy determined by a manager or the like, and causes the behavior optimization unit 20 to use a maximization model for the selected operation policy to generate decision support information at each place in accordance with a work timing of each medical worker. For the medical workers, this decision support information supports a behavior to be taken on each patient in order to maximize the overall efficiency of the hospital in line with the selected operation policy in accordance with a change in an environment of the hospital.

FIG. 5 is an illustration of an example of a processing flow of the behavior optimization unit 20 configured to provide decision support information for examination. In this flow, through use of the learning model of the selected operation policy, the patient attributes, the surrounding environment, and the like are used to notify the medical worker of an examination method maximizing the operation policy index as the optimum examination method. For this processing operation, an examination method optimized for a specified policy, for example, a policy prioritizing cost or effect, may be derived by individual machine learning based on past patient examination records having similar patient attributes.

Other decision support information for other purposes may also be provided based on the learning model of an operation policy having a similarly selected processing flow to be performed at the timing for providing decision support information for diagnosis, the timing for providing decision support information for treatment, or the timing for providing decision support information for discharge determination.

In FIG. 5, a handling flow for inpatients is illustrated. For other patients, the medical worker performing the work flow in the hospital may operate the behavior optimization unit 20 so that decision support information is appropriately provided in accordance with the work flow before performing work on an individual patient.

In many existing hospitals, medical workers determine and perform the optimum work (for example, examination or treatment) for a patient as appropriate. This can be called partial optimization of the examination plan, treatment plan, and the like. It is assumed that a medical worker provides the optimum treatment for a patient within the scope of the work that the medical worker is responsible for. However, the partial optimization for a patient in one department does not always give the optimal result for the patients and the hospital in terms of the entire hospital. To give a simple example, for a patient undergoing a plurality of examinations at a health checkup, even when the patient undergoes an expensive examination that only takes a short time for each individual examination, unless the next examination or diagnosis is immediately available, the patient will have to wait longer and will not return home earlier. Although capable of making satisfactory determinations when optimizing an individual event, humans are not suited to make optimized determinations of individual events based on overall resources. It is difficult to optimize individual events by further taking into account the hospital operation policy in addition to the above-mentioned perspective. In addition, the operation policy of the hospital may change.

In many cases, the operation policy of a hospital is determined in consideration of its management impact. Meanwhile, it is difficult for medical workers to determine whether their determinations and selections are in accordance with the hospital operation policy.

The above-mentioned medical information processing system, to which this invention is applied, helps to solve those problems. Specifically, medical workers are supported in the performance of their work in consideration of the impact on the running of the hospital, and as a result, work determinations that contribute to operation in accordance with the hospital operation policy can be made.

When explained in more detail based on ex-post facto analysis, behaviors relating to the following factors can be supported in this invention.

Avoidance of Partial Optimization:

Many departments are involved in the hospital work relating to one patient. It is difficult for humans to appropriately determine the overall optimal treatment rather than what is partially optimal for a given department, for each patient, each situation, and each resource situation. Medical workers are forced to rely on heuristics in complex and difficult situations.

Avoidance of Decrease in Management Efficiency:

It is difficult for humans to determine what kind of treatment is to be performed on the patient in front of the medical worker and whether that treatment is the best treatment for maximizing management efficiency in consideration of the entire hospital, for each patient and for each situation.

Avoidance of confusion in operation adjustment (change in management policy): For example, it is not possible to perform work that has been optimized as a whole by taking into consideration changes in an environment outside the hospital (for example, climate, establishment of competing hospital, seasonal changes in number of patients, resource situation of transfer destination hospital, and healthcare cost data), and deviation from the treatment plan (for example, emergency patient or shift from treatment plan). Even when the management index to be emphasized is changed, it is difficult for humans and conventional systems to present suggestions of the optimum treatment to medical workers.

Medical care has a high risk of prediction error, for example, a sudden change in a patient, and a uniform operation is difficult unlike in a factory of an industrial product. Moreover, a low risk is required for medical care. Meanwhile, administrators and managers issue general instructions to maximize the management policy of the entire hospital (for example, to shorten hospital stay time while maintaining unscheduled readmission rate, staff working hours, and patient satisfaction). In addition, it is difficult for medical workers in hospitals to adjust their work by incorporating management policies into their own work.

In this medical information processing system, for example, behavior support information for optimizing cost-effectiveness, total hospitalization time, and the like can be presented to each medical worker relating to each patient in accordance with various information on hospital resources at that time, patient ID, weather, and the like registered in a database.

For this reason, work adjustments reflecting the input operation policy together with a large amount of various data, such as climate, establishment of a competing hospital, seasonal changes in the number of patients, resource situation of the transfer destination hospital, and changes in healthcare costs, and deviation from the treatment plan or the like (for example, emergency patient or shift from treatment plan) can be performed to optimize overall resources (more efficient operation in terms of, for example, number of beds, staff capabilities, and unused examination equipment), which is difficult for humans.

As described above, the medical information processing system to which this invention is applied this invention can support decisions by medical workers with respect to each patient based on the selected operation policy information on the entire hospital and a database reflecting the current environment inside and outside the hospital.

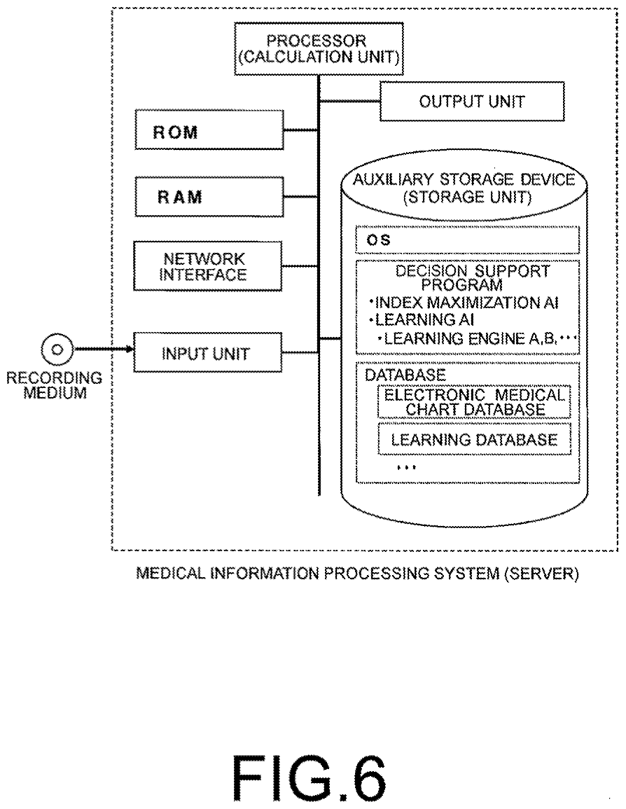

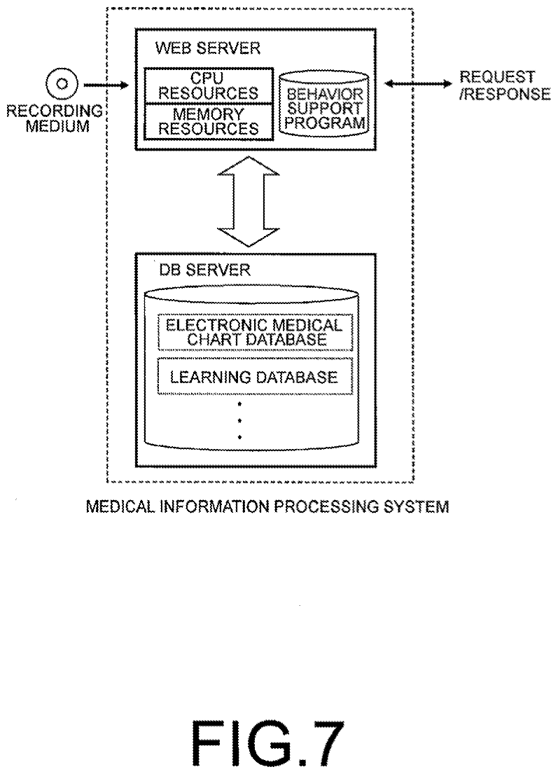

Each part of the system may be implemented by appropriately using a combination of the hardware and software of a computer system (server system) and virtualization technology, as illustrated in FIG. 6 and FIG. 7. The computer system includes one or more processors and memories tailored to the desired mode. In this computer system mode, each part may be implemented by a behavior support system program developed in the memory, in which hardware, for example, one or more processors is operated by an execution instruction group or a code group based on the program. In this case, the program may implement each part in cooperation with functions provided by software, such as an operating system, a microprogram, and a driver, as required.

The program data developed in the memory includes as appropriate an execution instruction group, a code group, a table file, content data, and the like that cause the processor to operate as one or more of the above-mentioned units.

The computer system is not required to be constructed as a single device, and may be constructed as a so-called thin client, distributed computing, or cloud computing by combining a plurality of servers, computers, virtual machines, and the like.

A part or all of the computer system may be replaced with hardware or firmware (e.g., one or more of large-scale integration (LSI), field programmable gate array (FGPA), a combination of electronic elements). Similarly, only a portion of each part may be replaced with hardware or firmware.

The program may be recorded in a non-transitory manner on a recording medium and distributed. The program recorded on the recording medium is read into the memory in a wired manner, in a wireless manner, or via the recording medium itself, and operates the processor and the like.

In this specification, the term “recording medium” includes similarly-termed storage media, memory devices, storage devices, and the like. Examples of the recording medium include an optical disc, a magnetic disk, a semiconductor memory device, a hard disk device, and a tape medium. It is desired that the recording medium be non-volatile. The recording medium may be a combination of a volatile module (e.g., random access memory (RAM)) and a nonvolatile module (e.g., read only memory (ROM)).

Stating the above-mentioned embodiment another way, the medical information processing system according to this invention can be implemented by causing an information processing system configured to operate as a medical information processing system to operate as an input/output unit, a behavior optimization unit, and a model learning unit based on a behavior support program developed in a memory.

Similarly, stating the above-mentioned embodiment another way, the medical information processing system according to this invention can be constructed by a recording medium including a behavior support program configured to be expanded in a memory and be operated by a processor of the information processing system, the recording medium being configured to cause an information processing resource to execute a learning step, an input step, and a behavior support step in a timely manner.

Example embodiments of this invention have been described as an example. However, specific configurations of this invention are not limited to the above-mentioned example embodiments, and changes without departing from the gist of the invention are also included in this invention. For example, changes such as separation and merging of the block components and a switch of processing steps in the above-mentioned example embodiments can be freely carried out as long as the purport and the above-mentioned functions of this invention are satisfied, and the above description does not limit this invention.

This application is based upon and claims the benefit of priority from Japanese patent application No. 2017-172847, filed on Sep. 8, 2017, the disclosure of which is incorporated herein in its entirety by reference.

### REFERENCE SIGNS LIST

1 medical information processing system (computer system)
10 input/output unit
20 behavior optimization unit
30 model learning unit

1. A medical information processing system, comprising:
an operation policy input unit configured to receive, as a selected operation policy, one selected from a plurality of management policies takable by an entire hospital;
a model learning unit configured to generate, for each selectable operation policy, a maximization model in response to a change in an environment of the hospital for each operation policy by referring to a database managed so as to be accessible at the hospital; and
a behavior optimization unit configured to generate, through use of the maximization model for the selected operation policy, decision support information for a medical worker with respect to each patient in order to maximize overall efficiency of the hospital in accordance with the change in the environment of the hospital.

2. The medical information processing system according to claim 1,
wherein the model learning unit is configured to generate, for each selectable operation policy, the maximization model in response to the change in the environment of the hospital and a shift from a treatment plan with respect to each patient by referring to the database, and
wherein the behavior optimization unit is configured to generate, through use of the maximization model for the selected operation policy, the decision support information for the medical worker with respect to each patient in order to maximize overall efficiency of the hospital in accordance with the change in the environment of the hospital and the shift.

3. The medical information processing system according to claim 2, wherein the behavior optimization unit is configured to generate, through use of the maximization model for the selected operation policy, a notification indicating a change policy of a hospital room environment with respect to each patient as the decision support information in accordance with a current situation of the database.

4. The medical information processing system according to claim 1, wherein the behavior optimization unit is configured to generate, through use of the maximization model for the selected operation policy, a notification indicating an ordering policy of each patient as the decision support information in accordance with a current situation of the database.

5. The medical information processing system according to claim 1, wherein the behavior optimization unit is configured to generate, through use of the maximization model for the selected operation policy, a notification indicating a post-discharge care work policy for each patient as the decision support information in accordance with a current situation of the database.

6. The medical information processing system according to claim 1,
wherein the change in the environment of the hospital includes at least one of pieces of information on a number of inpatients at a neighboring medical institution, a number of inpatients at a transfer destination medical institution, a local climate, a local population change, and a number of outpatients, and
wherein the change in the environment of the hospital is reflected by the model learning unit in the maximization model for each operation policy by managing each change in the environment of the hospital as an individual item in the database.

7. The medical information processing system according to claim 2,
wherein the treatment plan with respect to each patient includes a scheduled date and an execution date each of which relates to at least any one of an examination date, an informed consent date, and a discharge date, and
wherein the treatment plan with respect to each patient is reflected by the model learning unit in the maximization model for each operation policy by managing each of the scheduled date and the execution date of the treatment plan with respect to each patient as an individual item in the database.

8. The medical information processing system according to claim 2,
wherein the shift includes at least an occurrence situation of an emergency patient and a degree of recovery of the patient, and
wherein the shift is reflected by the model learning unit in the maximization model for each operation policy by managing each shift from the treatment plan with respect to each patient as an individual item in the database.

9. A determination support information generation method, comprising:
preliminarily generating, by a model learning unit, for each of a plurality of operation policies takable by the entire hospital, a maximization model in response to a change in an environment of a hospital for each operation policy by referring to a database managed so as to be accessible at the hospital;
receiving, by an operation policy input unit, one selected from the plurality of operation policies, as a selected operation policy; and
generating, by a behavior optimization unit, decision support information for a medical worker with respect to each patient in order to maximize overall efficiency of the hospital in accordance with the change in an environment of the hospital through use of the maximization model for the selected management policy.

10. A non-transitory computer readable recording medium for storing a program to cause a processor of an information processing system to operate as:
an operation policy input unit configured to receive, as a selected operation policy, one selected from a plurality of operation policies takable by an entire hospital;
a model learning unit configured to generate, for each selectable operation policy, a maximization model in response to a change in an environment of the hospital for each operation policy by referring to a database managed so as to be accessible at the hospital; and
a behavior optimization unit configured to generate, through use of the maximization model for the selected operation policy, decision support information for a medical worker with respect to each patient in order to maximize overall efficiency of the hospital in accordance with the change in an environment of the hospital.

11. The determination support information generation method according to claim 9,
generating, by the model learning unit, for each selectable operation policy, the maximization model in response to the change in the environment of the hospital and a shift from a treatment plan with respect to each patient by referring to the database, and
generating, by the behavior optimization unit, through use of the maximization model for the selected operation policy, the decision support information for the medical worker with respect to each patient in order to maximize overall efficiency of the hospital in accordance with the change in the environment of the hospital and the shift.

12. The determination support information generation method according to claim 11,
generating, by the behavior optimization unit, through use of the maximization model for the selected operation policy, a notification indicating a change policy of a hospital room environment with respect to each patient as the decision support information in accordance with a current situation of the database.

13. The determination support information generation method according to claim 9,
generating, by the behavior optimization unit, through use of the maximization model for the selected operation policy, a notification indicating an ordering policy of each patient as the decision support information in accordance with a current situation of the database.

14. The determination support information generation method according to claim 9,
generating, by the behavior optimization unit, through use of the maximization model for the selected operation policy, a notification indicating a post-discharge care work policy for each patient as the decision support information in accordance with a current situation of the database.

15. The determination support information generation method according to claim 9,
wherein the change in the environment of the hospital includes at least one of pieces of information on a number of inpatients at a neighboring medical institution, a number of inpatients at a transfer destination medical institution, a local climate, a local population change, and a number of outpatients, and
wherein the change in the environment of the hospital is reflected by the model learning unit in the maximization model for each operation policy by managing each change in the environment of the hospital as an individual item in the database.

16. The determination support information generation method according to claim 12,
wherein the treatment plan with respect to each patient includes a scheduled date and an execution date each of which relates to at least any one of an examination date, an informed consent date, and a discharge date, and
wherein the treatment plan with respect to each patient is reflected by the model learning unit in the maximization model for each operation policy by managing each of the scheduled date and the execution date of the treatment plan with respect to each patient as an individual item in the database.

17. The determination support information generation method according to claim 12,
wherein the shift includes at least an occurrence situation of an emergency patient and a degree of recovery of the patient, and
wherein the shift is reflected by the model learning unit in the maximization model for each operation policy by managing each shift from the treatment plan with respect to each patient as an individual item in the database.

18. The non-transitory computer readable recording medium according to claim 10,
wherein the model learning unit is configured to generate, for each selectable operation policy, the maximization model in response to the change in the environment of the hospital and a shift from a treatment plan with respect to each patient by referring to the database, and
wherein the behavior optimization unit is configured to generate, through use of the maximization model for the selected operation policy, the decision support information for the medical worker with respect to each patient in order to maximize overall efficiency of the hospital in accordance with the change in the environment of the hospital and the shift.

19. The non-transitory computer readable recording medium according to claim 18, wherein the behavior optimization unit is configured to generate, through use of the maximization model for the selected operation policy, a notification indicating a change policy of a hospital room environment with respect to each patient as the decision support information in accordance with a current situation of the database.

20. The non-transitory computer readable recording medium according to claim 10, wherein the behavior optimization unit is configured to generate, through use of the maximization model for the selected operation policy, a notification indicating an ordering policy of each patient as the decision support information in accordance with a current situation of the database.
